(19) United States
(12) Patent Application Publication
(10) Pub. No.: US 2010/0069997 A1
(43) Pub. Date: Mar. 18, 2010

(54) NEUROSTIMULATION APPARATUS

(75) Inventor: Denis Dupeyron, Mougins (FR)

Correspondence Address: MARSH, FISCHMANN & BREYFOGLE LLP, 8055 East Tufts Avenue, Suite 450, Denver, CO, 80237, US

(73) Assignee: Otologics, LLC, Boulder, CO (US)

(21) Appl. No.: 12/211,643

(22) Filed: Sep. 16, 2008

(51) Int. Cl. A61N 1/02

(52) U.S. Cl. 607/57

(57) ABSTRACT

An output stage for an auditory neurostimulation electrode and related system arrangements and methods are provided. The output stage is operable to effect a plurality of stimulation and discharge intervals, and includes a stimulation channel and a discharge channel coupled to the electrode, wherein a stimulation current and a discharge current may flow therethrough during the corresponding stimulation intervals and discharge intervals. The output stage also includes a controller that is operable to selectively control the flow of current through the stimulation channel and the discharge channel during the stimulation and discharge intervals. Further, one of the stimulation channel and the discharge channel couples the electrode to a single voltage supply, and the other of the stimulation channel and the discharge channel couples the electrode to a reference potential node. The output stage is intrinsically capable of maintaining an equilibrium of charges and does not require any complex control means to equilibrate the charges on the electrode.

### FIELD OF THE INVENTION

The present invention relates to neurostimulation, and more particularly to an output stage for signal generation componentry of a neurostimulation implant device. The invention is particularly apt for auditory neurostimulation applications, and reduces the power and volume requirements in such applications.

### BACKGROUND OF THE INVENTION

The utilization of neurostimulation implant devices is ever-increasing. Such devices utilize a plurality of implanted electrodes that are selectively activated to affect a desired neuro-response, including sound sensation, pain/tremor management, and urinary/anal incontinence. By way of primary interest, auditory neurostimulation implant devices include auditory brainstem implant (ABI) and cochlear implant (CI) devices.

In the case of CI devices, an electrode array is inserted into the cochlea of a patient, e.g. typically into the scala tympani so as to access and follow the spiral curvature of the cochlea. The array electrodes are selectively driven to stimulate the patient's auditory nerve endings to generate sound sensation. In this regard, a CI electrode array works by utilizing the tonotopic organization, or frequency-to-location mapping, of the basilar membrane of the inner ear. In a normal ear, sound vibrations in the air are transduced to physical vibrations of the basilar membrane inside the cochlea. High frequency sounds do not travel very far along the membrane, while lower frequency sounds pass further along. The movement of hair cells, located along the basilar membrane, creates an electrical disturbance, or potential, that can be picked up by auditory nerve endings that generate electrical action pulses that travel along the auditory nerve to the brainstem. In turn, the brain is able to interpret the nerve activity to determine which area of the basilar membrane is resonating, and therefore what sound frequency is being sensed. By directing which electrodes of a CI electrode array are activated, cochlear implants can selectively stimulate different parts of the cochlea and thereby convey different acoustic frequencies corresponding with a given audio input signal.

With ABI systems a plurality of electrodes may be implanted at a location that bypasses the cochlea. More particularly, an array of electrodes may be implanted at the cochlea nucleus, or auditory cortex, at the base of the brain to directly stimulate the brainstem of a patient. Again, the electrode array may be driven in relation to the tonotopic organization of a recipient's auditory cortex to obtain the desired sound sensation.

As may be appreciated, in the case of either ABI electrodes or CI electrodes, audio signals (e.g. from a microphone) may be processed, typically utilizing what is referred to as a speech processor, to generating stimulation signals utilized to selectively drive the electrodes for stimulated sound sensation. Further, in both implant approaches, a source of power may be included to power the stimulation signal generator.

Neurostimulation generally provides a system that recovers any charges that are injected into a patient's body through the electrodes (i.e., “equilibrating charges”), so that accumulated charges do not remain in the tissue of a patient. To accomplish this, subsequent to each stimulation interval with a predetermined level of electrical current for a predetermined time period, the same level of electrical current for the same time period may be applied in the opposite direction. That is, a plurality of biphasic pulses (i.e., stimulation pulses and discharge pulses) may be delivered to a patient's tissue through the electrode array. Any difference between the ideal discharge and the actual discharge results in a disruptive leakage current.

### SUMMARY OF THE INVENTION

In view of the foregoing, a primary objective of the present invention is to provide an output stage for an auditory neurostimulation electrode that receives power from a single power supply.

An additional objective of the present invention is to provide an output stage for an auditory neurostimulation electrode that has a relatively small volume and low power requirements.

A further objective of the present invention is to provide an output stage for an auditory neurostimulation electrode that is intrinsically capable of maintaining an equilibrium of charges without requiring complex control and monitoring means.

One or more of the above-noted objectives and additional advantages are realized by an output stage of the present invention. The output stage for an auditory neurostimulation electrode that is operable to effect a plurality of stimulation and discharge intervals may include a stimulation channel and a discharge channel, each coupled to the electrode, wherein a stimulation current may flow through the stimulation channel during a stimulation interval, and a discharge current may flow through the discharge channel during a discharge interval. The output stage may further include a controller that is operable to selectively control the flow of current through the stimulation channel and the discharge channel during the stimulation and discharge intervals, respectively. Additionally, one of the stimulation channel and the discharge channel may couple the electrode to a voltage supply, and the other of the stimulation channel and the discharge channel may couple the electrode to a reference potential node. In this regard, the present invention provides an output stage that is intrinsically capable of maintaining an equilibrium of charges, operates using a single power supply, and does not require complex control or monitoring means.

In one aspect, the controller may be operable to control the timing of the stimulation and discharge intervals such that the intervals are successive. Furthermore, the controller may be operable to selectively adjust the magnitude of the stimulation and discharge currents. As can be appreciated, these features may be advantageous as they provide the ability to selectively adapt a neurostimulation system to the needs of a particular patient.

In one aspect, the amount of charge transferred during a discharge interval is greater than the amount of charge transferred during a stimulation interval. In this regard, the output stage may be intrinsically capable of maintaining an equilibrium of charges and may operate to remove the charges from the tissue of a patient each stimulation/discharge cycle. In one embodiment, this is accomplished by providing components in the stimulation and discharge channels that are sized to possess certain desirable conductive properties. For example, the stimulation channel and the discharge channel may each include one or more transistors (e.g., a MOSFET, a bipolar junction transistor, or the like) whose relative physical dimensions (e.g., channel length, channel width, etc. . . . ) are chosen so that the charges transferred during the discharge interval are slightly greater than the charges transferred during the stimulation interval.

In a related aspect, the amount of charges that are transferred during a stimulation interval and a discharge interval may be determined by corresponding stimulation and discharge current mirrors. In this regard, physical properties of the various components (e.g., transistors) of the current mirrors may be chosen to provide suitable stimulation and discharge currents.

In another aspect, the output stage may include a charge recovery mechanism that is operable to recover accumulated charges from an electrode. For example, in one embodiment, a resistor is provided that is selectively interconnectable between an electrode and a reference potential node, such that the controller may selectively cause the accumulated charges to be removed from the electrode at a desirable time (e.g., when a patient turns the neurostimulation apparatus off at night).

In yet another aspect, the output stage may be interconnected with an electrode interface that is operable to selectively interconnect an output of the output stage to one or more of a plurality of auditory neurostimulation electrodes. In one embodiment, the electrode interface is operable to selectively interconnect the output of the output stage to a first and second set of the plurality of auditory neurostimulation electrodes to effect a plurality of successive stimulation and discharge intervals on the first and second sets of electrodes. Further, the first and second sets of electrodes may not be identical. For example, the first set of electrodes may include the electrodes e₁, e₂, and e₃, while the second set may include the electrodes e₃, e₄, e₅, and e₆.

In another embodiment, a method for driving an electrode for auditory neurostimulation is provided. The method may include first transferring a stimulation current between an electrode and one of a voltage supply and a reference potential node. Further, the method may include second transferring a discharge current between the electrode and the other of the voltage supply and the reference potential node. In this regard, a method for driving an auditory neurostimulation electrode that utilizes a single power supply is provided.

Various features and refinements to the above-noted method may also be provided. For example, in one embodiment, the amount of charge transferred in the first transferring step may be less than the amount of charge transferred in the second transferring step. Further, the method may also include limiting the amount of charge transferred in the second transferring step dependent upon the voltage potential on the electrode.

In another aspect, the method may include selectively alternating between the first and second transferring steps to provide auditory neurostimulation to a patient. Additionally, the amount of charge transferred and the duration of each transferring step may be selectively varied. This may be accomplished by providing a controller, or by providing components (e.g., transistors) whose conductive properties are dependent upon their respective physical dimensions. In one embodiment, current mirrors that are coupled to the electrode may provide the current for each transferring step.

In a related aspect, the method may include removing accumulated charges from the electrode. This step may be performed at any desirable time. In one embodiment, the accumulated charges are removed when a patient turns an implant device off.

Additional aspects and corresponding advantages will be apparent to those skilled in the art upon consideration of the further description that follows.

### BRIEF DESCRIPTION OF THE DRAWINGS

FIG. 1 is a schematic illustration of one system embodiment comprising the present invention.

FIG. 2a is a schematic illustration of the present invention during a stimulation interval.

FIG. 2b is a schematic illustration of the present invention during a discharge interval.

FIG. 3 is a graphical illustration of current flow and accumulated charges on an electrode that is coupled to an output stage of the present invention.

FIG. 4 is a graphical illustration of the leakage current over time for an electrode that is coupled to an output stage of the present invention.

FIG. 5 is a schematic illustration of another embodiment of the present invention that includes a charge recovery mechanism.

FIG. 6 is another schematic illustration of one system embodiment comprising the present invention.

### DETAILED DESCRIPTION OF THE INVENTION

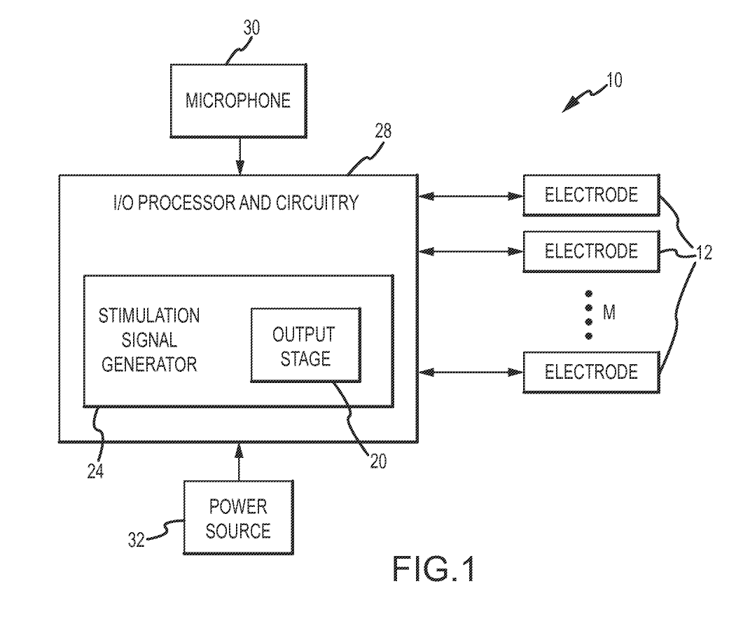

FIG. 1 illustrates one embodiment of an auditory neurostimulation system 10 comprising the present invention. Variations in the system 10 and other neurostimulation applications will be apparent to those skilled in the art.

As shown in FIG. 1, the auditory neurostimulation system 10 may include an array of M electrodes 12 (where M is an integer greater than or equal to one) that are electrically interconnected to an input/output (I/O) processor and circuitry 28. The I/O processor and circuitry 28 may include a stimulation signal generator 24 for generating electrode stimulation signal(s) that are delivered to a patient through the electrodes 12. The stimulation signal generator 24 may further include an output stage 20 that is operable to electrically drive the electrodes 12 to deliver biphasic stimulation and discharge intervals to the tissue of a patient. Power may be provided to the output stage by a single power supply. The specific operation of the output stage 20 is discussed in detail below. Operation of the stimulation signal generator 24 may be responsive to audio input signals received at the I/O processor and circuitry 28, as generated by a microphone 30.

As further shown in FIG. 1, the auditory neurostimulation system 10 may also comprise a power source 32 interconnected to the I/O processor and circuitry 28 for directing power thereto. The power source 32 may comprise various diodes, capacitors, inductors, or other components to rectify an AC signal to DC power, or any other type of AC-to-DC and/or DC-to-DC converter.

The embodiment shown in FIG. 1 may be provided and controlled to provide for monopolar stimulation, common ground stimulation or bipolar stimulation. For example, one electrode (e₁) from the set of M electrodes 12 may be selected under the control of the I/O processor and circuitry 28. Current may be provided to the electrode e₁ by the output stage 20 with a current return path through an electrical reference electrode. This mode of stimulation is referred to as “monopolar.” Alternatively, if one electrode (e₂) from the set of M electrodes is selected to provide stimulation current and the remaining electrodes in the set of M electrodes are electrically connected to the electrical reference, then this mode of stimulation is referred to as “common ground.” Finally, if two electrodes (e₁ and e₂) from the set of M electrodes are selected to provide stimulation such that in an alternating manner the first electrode e₁ is electrically connected to the stimulation current source (i.e., the output stage 20) and e₂ is electrically connected to the electrical reference and subsequently e₂ is electrically connected to the stimulation current source and e₁ is electrically connected to the electrical reference, then this stimulation mode is known as “bipolar stimulation.” In all of these stimulation schemes, balanced anodic and cathodic stimulation may be provided.

Further, the embodiment may provide for simultaneous stimulation or pulsatile (e.g. non-simultaneous) stimulation. For example, under the control of the I/O processor and circuitry 28, two of the electrodes 12 may be selected to provide stimulation current such that unequal amounts of stimulation current are provided by the two electrodes (e.g., the current magnitudes are different). This bias in stimulation current will create an intermediate pitch perception for the patient between the two electrodes. The tonotopic location of the pitch perception can be controlled by the bias in the current between the two electrodes.

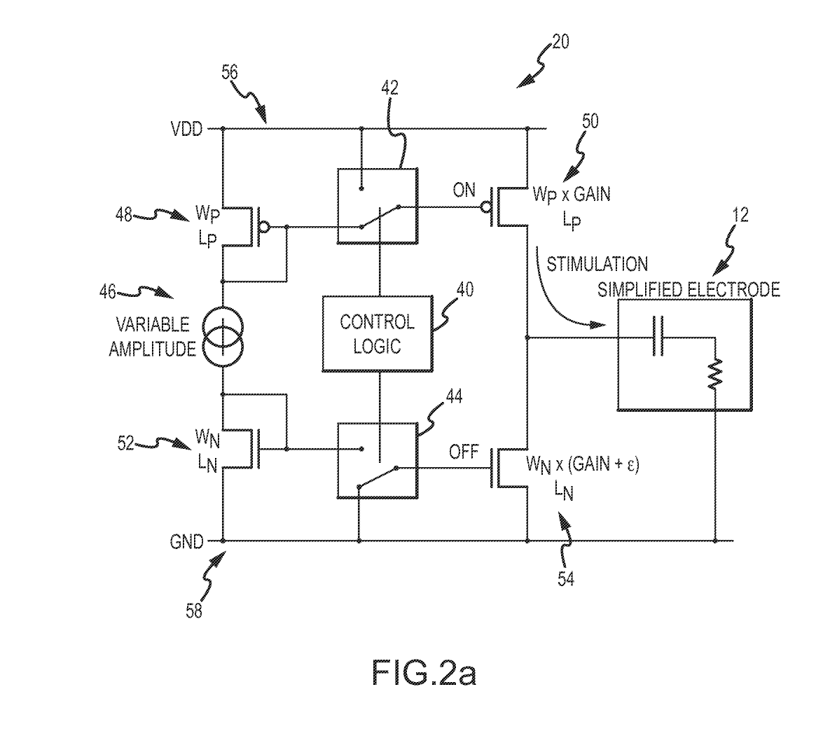

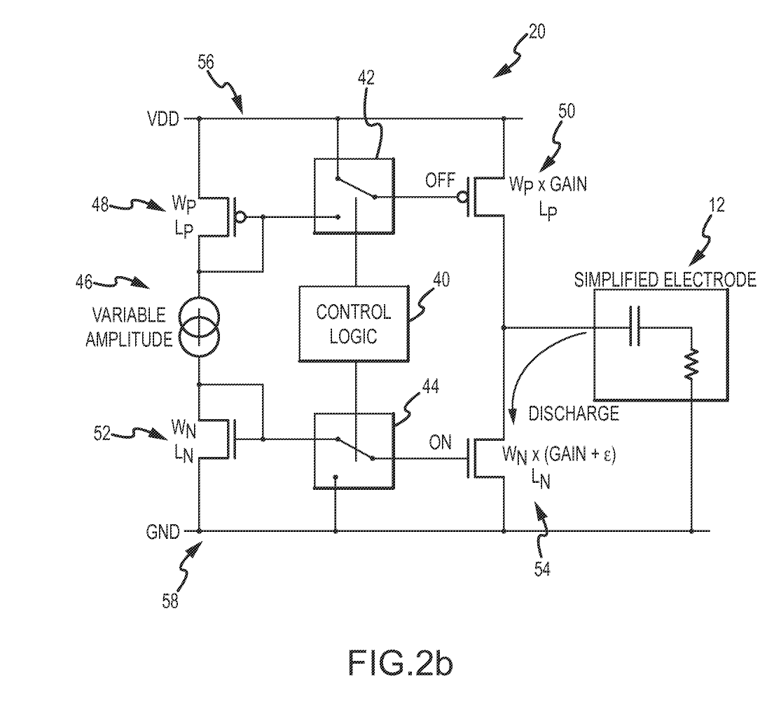

Reference is now made to FIGS. 2a, 2b, 3, 4, and 5, which illustrate various embodiments and operational characteristics of the output stage 20 in accordance with the present invention.

In particular, FIGS. 2a-2b illustrate schematic illustrations of one embodiment of an output stage 20 that is powered by a single power supply during a stimulation interval (FIG. 2a) and a discharge interval (FIG. 2b) of a neurostimulation sequence. Referring to FIG. 2a, a simplified representation of an electrode 12 is shown that includes a capacitor and resistor connected in series. To provide electrical current for a stimulation interval, the electrode 12 is connected to a stimulation MOSFET current mirror (or current source) that includes two p-channel MOSFET transistors 48 and 50 and a controllable switch 42 that is operable to selectively activate and deactivate the stimulation current mirror. During a stimulation interval, current flows through a stimulation channel formed by the transistor 50 from an output voltage VDD 56 of a single power supply to the electrode 12. Similarly, to effect a discharge interval, the electrode 12 is further connected to a discharge MOSFET current mirror that includes two n-channel MOSFET transistors 52 and 54, and a switch 44 that is operable selectively activate and deactivate the discharge current mirror. During a discharge interval, current flows through a discharge channel formed by the transistor 54 from the electrode 12 to a ground node 56. As discussed in further detail below, the timing and control of the switches 42 and 44 is provided by control logic 40, which may include any combination of software and hardware componentry.

The operation of the stimulation and discharge current mirrors is now described. In this embodiment, the core of the stimulation current mirror is the transistor 48 whose drain is shorted to its gate (i.e., diode connected) and thus operates in the saturation region. The current through the transistor 48 is provided by a connection between its source and the voltage VDD of the single power supply and a variable amplitude current source 48 (or current sink). When the switch 42 is in the position shown in FIG. 2a, the gates of the transistors 48 and 50 are electrically coupled together. In this regard, since the transistor 50 has the same gate-to-source voltage (V_{GS}) as the transistor 48, the current through the transistor 48 functions as a reference current (I_{REF}) for the output current (I_{O}) that passes through the transistor 50 and is delivered to the electrode 12 as a stimulation pulse. More particularly, the stimulation current through the transistor 50 will be related to the reference current through the transistor 48 by the ratio of the aspect ratios of the channels of the two transistors; that is, the relationship of the reference current to the stimulation current is solely determined by the geometry of the transistors 48 and 50. As labeled in FIG. 2a, the width of the channel of the transistor 48 is Wₚ and the length is Lₚ. Similarly, the width of the channel of the transistor 50 is (Wₚ+gain), and the length is Lₚ. The equation for the stimulation output current (I_{O}) as a function of the reference current (I_{REF}) is shown in Equation (1):

$${I}_{O} = {I}_{REF} \times \frac{( ( {W}_{P} \times gain ) / {L}_{P} )}{( {W}_{P} / {L}_{P} )} = {I}_{REF} \times gain \qquad ( 1 )$$

From Equation (1), it should be appreciated that the relative magnitude of the output current may be designed by sizing the dimensions of the transistors 48 and 50 accordingly. Further, the absolute magnitude of the currents may be controlled by the variable amplitude current source 46.

Subsequent to the stimulation current mirror is utilized to deliver a stimulation interval to the electrode 12, the discharge current mirror may be utilized to equilibrate the charges on the electrode 12 by applying a current in the opposite direction. This discharge interval is graphically illustrated in FIG. 2b. As shown, the control logic 40 has toggled the switch 42 to a position that deactivates the stimulation current mirror by connecting the gate of the transistor 50 to VDD. Further, the control logic 40 has activated the discharge current mirror by toggling the switch 44 to couple the gates of the transistors 52 and 54 together. The operation of the discharge current mirror is similar to the operation of the stimulation current mirror described above. That is, the magnitude of the discharge output current through the transistor 54 is related to the reference current through the transistor 52 by the ratio of the aspect ratios of the channels of the two transistors. The equation for the relationship between the output current (I_{O}) and the reference current (I_{REF}) for the discharge current mirror is shown in Equation (2):

$${I}_{O} = {I}_{REF} \times \frac{( ( {W}_{N} \times ( gain + ɛ ) ) / {L}_{N} )}{( {W}_{N} / {L}_{N} )} = {I}_{REF} \times ( gain + ɛ ) \qquad ( 2 )$$

As indicated by the presence of εE, the gain of the discharge current mirror may be designed to be slightly larger than the gain of the stimulation current mirror, such that the discharge current is slightly larger than the stimulation current. As discussed further below, this is to ensure system stability.

The control logic 40 may be operable to control the timing of the stimulation intervals and discharge intervals by selectively toggling the switches 42 and 44. In this regard, the control logic 40 may include any combination of software and hardware. Further, the control logic 40 may be hard coded or programmable by a patient or a technician. For example, it may be desirable to selectively adjust the duration of each stimulation-discharge cycle or the period between cycles to provide the best performance to a patient. Similarly, the variable amplitude current source 46 may be controllable by a patient or a technician. In this regard, it may be desirable to increase or decrease the magnitude of the neurostimulation to provide the optimum performance. In the case where the control logic 40 or the current source 46 is programmable, a suitable user interface may be provided.

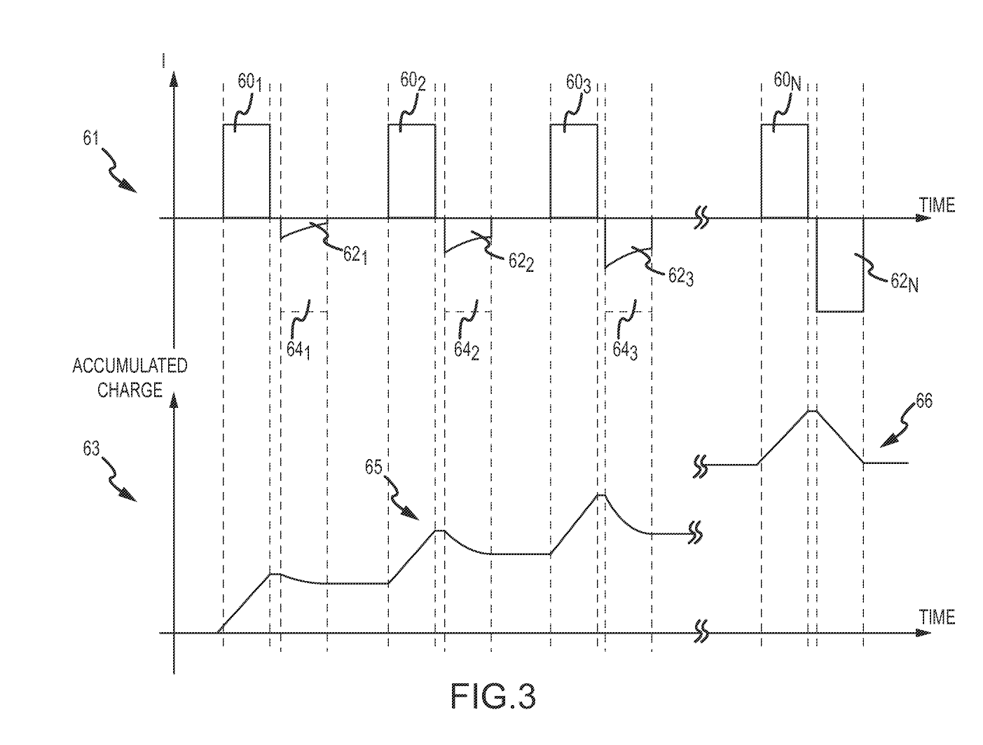

FIG. 3 illustrates graphs of the stimulation and discharge currents (graph 61) and the accumulated charges on an electrode (graph 63) during the start of a neurostimulation sequence (e.g., when a neurostimulation apparatus is first turned on in the morning). In operation, the initial stimulation pulses 60₁₋₃ are delivered to an electrode (e.g., the electrode 12 shown in FIGS. 2a-2b) by activating and deactivating the stimulation current mirror as described above in relation to FIG. 2a. A short time after each stimulation pulse 60, a discharge pulse 62 is initiated to recover the charges delivered to the electrode 12. As shown, the initial discharge pulses 62₁₋₃ are less than the desired discharge pulses 64₁₋₃ that would be required to fully discharge the accumulated charges from the electrode 12. This is due to the operational characteristics of the transistor 54 of the discharge current mirror shown in FIGS. 2a-2b. In the above description of the operation of the discharge current mirror, it was assumed that the transistor 54 was operating in saturation, which is required for the transistor to supply a constant-current output. To operate in saturation, the voltage at the drain of the transistor 54 (i.e., the voltage at the electrode 12) must be above a certain level (i.e., at least as great as the voltage on the gate of the transistor 54 (V_{GS}) minus the threshold voltage (Vₜ)). As can be appreciated, when the system is first turned on, the voltage on the electrode 12 will not be sufficient for the transistor 54 to operate in saturation mode, thereby causing the actual discharge pulses 62₁₋₃ to be less than the desired discharge pulses 64₁₋₃.

This initial difference between the magnitudes of the stimulation pulses and the discharge pulses will cause the rest voltage, the voltage potential that the electrode returns to after the completion of a stimulation-discharge cycle, to increase slowly due to the accumulation of charges that are not discharged from the electrode. The portion of the graph 63 indicated by an arrow 65 illustrates this effect of accumulating charges. It should be noted that as the rest voltage on the electrode increases, the discharge pulses will also increase due to in the increased voltage at the drain (i.e., “headroom”) of the transistor 54. As the system reaches stead-state (e.g., a few tens of stimulation-discharge cycles and typically much less than one second), the stimulation pulses 60_{N} and discharge pulses 62_{N} will both be at their desired magnitudes, and the rest voltage will have reached a stable level that permits both transistors 50 and 54 to operate in saturation mode, as shown in the portion of the graph 63 indicated by the arrow 66.

As discussed above, the output stage 20 may be designed such that the discharge current is slightly larger than the stimulation current when the system is operating in steady-state. This feature may be achieved by sizing the transistors of the aforementioned current mirrors accordingly. The primary purpose for this design is to provide a simple solution for ensuring system stability. As can be appreciated, when the discharge current is slightly greater than the stimulation current, the rest voltage on the electrode will tend to decrease since the charges removed from the electrode each cycle are greater than the charges delivered to the electrode. However, if the rest voltage is decreased to a point where the transistor 54 does not have enough headroom to fully operate in saturation mode, then the discharge current will automatically be reduced to a level that is below the stimulation current, which causes the rest voltage on the electrode to increase. Thus, the present design provides for a simple automatic feedback mechanism to ensure that the system remains intrinsically stable. Notably, this design does not require any intricate monitoring and control means to ensure that the charges are equilibrated, which reduces the hardware required, the power consumed, and the complexity of the design.

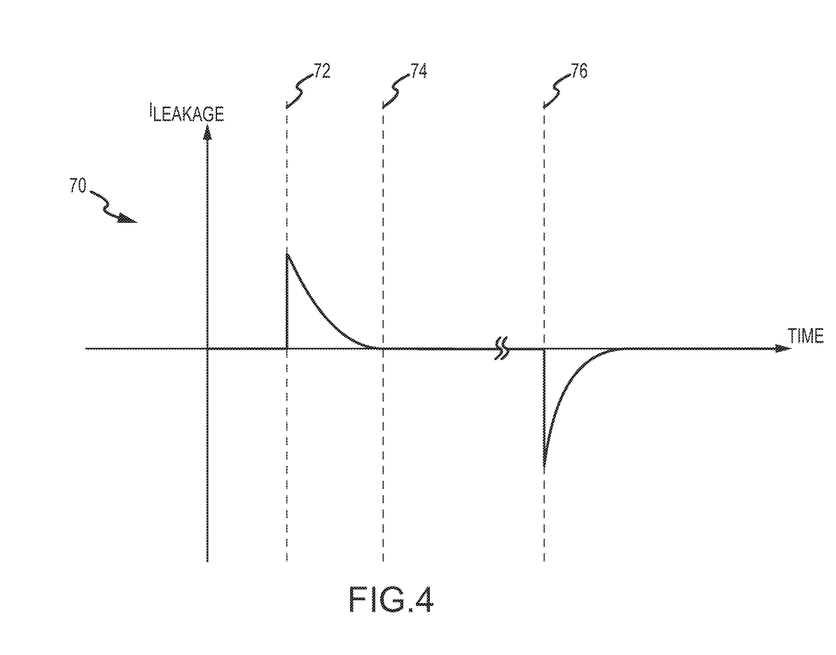

FIG. 4 is a graph 70 of the equivalent leakage current for a neurostimulation apparatus of the present invention when the apparatus is first turned on, during steady-state operation, and when the apparatus is turned off. Initially, the neurostimulation apparatus is turned on at a time indicated by the dashed line 72. As can be seen, the leakage current is initially present but decreases rapidly as the rest voltage of the electrode increases (See FIG. 3), thereby permitting the discharge current mirror to more fully remove the accumulated charges from the electrode. When the rest voltage is high enough for the discharge current mirror to operate fully (i.e., the time indicated by the dashed line 74), the leakage current virtually disappears. That is, the equivalent leakage current is only transient (e.g., much less than one second), and virtually no DC leakage current exists. This feature is desirable as a DC leakage current may be damaging to a patient's tissue and may also reduce the performance of the neurostimulation apparatus.

In one embodiment of the present invention, a charge recovery mechanism is provided to recover the charges on an electrode that are present due to the initial transient leakage current. The charge recovery mechanism may be operable to remove the accumulated charges periodically, when the apparatus is turned off, or any other desirable time. The effect of the charge recovery mechanism on the equivalent leakage current is shown in the graph 70 at the time indicated by the dashed line 76. As can be seen, substantially all of the charges that accumulated when the apparatus was turned on at time 72 are then recovered at time 76 so that virtually no disruptive charges remain in the tissue of a patient.

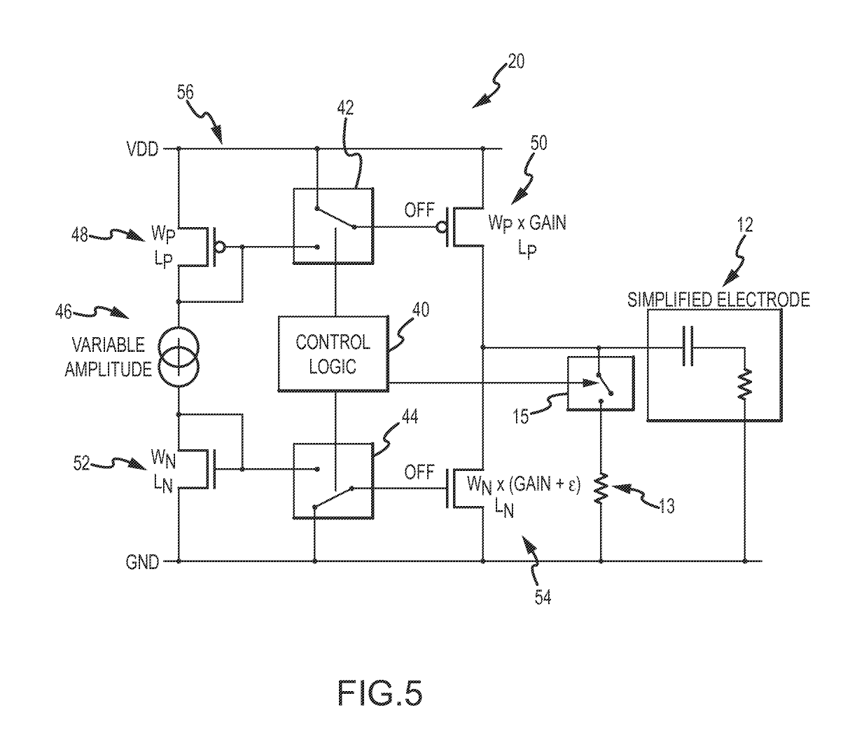

FIG. 5 illustrates one embodiment of an output stage 20 that includes a charge recovery mechanism. As shown, a controllable switch 15 (e.g., a transistor), interconnected to the control logic 40, is provided to selectively couple the electrode 12 to ground through a resistor 13. In operation, the control logic 40 may be operable to toggle the switch 15 at a time when the accumulated charges on the electrode 21 are to be removed (e.g., when the apparatus is turned off by the patient at night). In this regard, the accumulated charges may flow through the resistor 13 to ground to remove them from the tissue of a patient. As can be appreciated, the resistor 13 may be suitably chosen such that a desirable magnitude of current will flow as the charges are being recovered. Further, other techniques may be used to accomplish the task of recovering charges from the electrode 12. Those other techniques may include more sophisticated methods for regulating the discharge current, which may be desirable in certain instances.

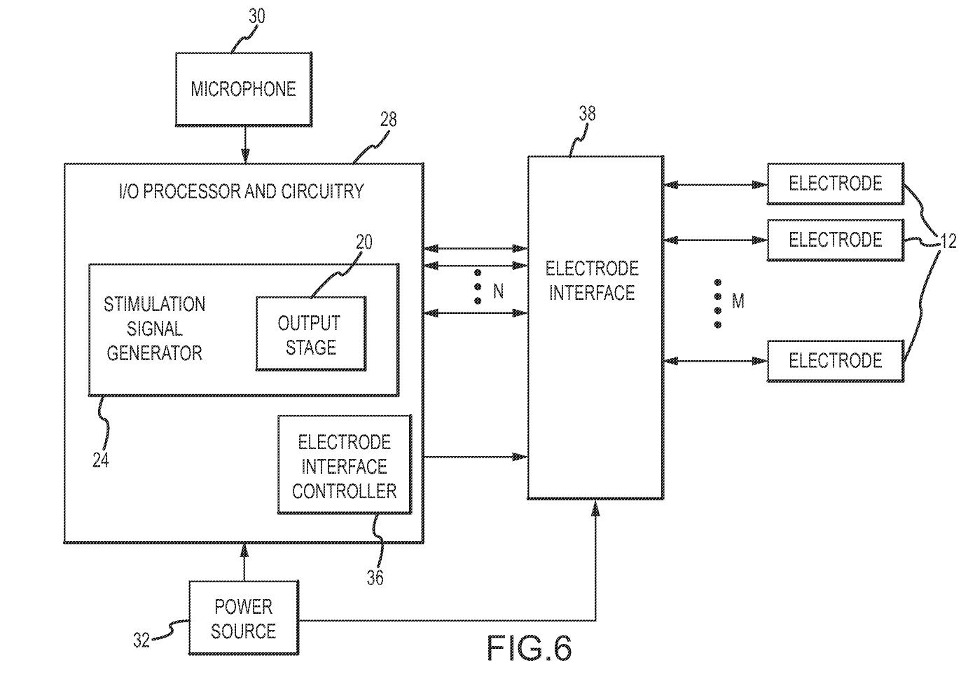

FIG. 6 is another schematic illustration of one system embodiment comprising the present invention. In this embodiment, an electrode interface 38 may be provided that is operable to electrically interconnect M electrodes 12 to N stimulation signal channels. In this regard, the electrode interface 38 may be selectively controllable to route one or more stimulation signals received from one or more of the N stimulation channels to one or more of the M electrodes 12 where the signals may be employed for neurostimulation. Further, the electrode interface 38 may be provided so as to route one or more electrode stimulation signals as current signals without changing the amplitude, frequency, or width of pulses comprising the current signal, and without otherwise buffering the current signal(s).

For the purpose of controlling the electrode interface 38, the I/O processor and circuitry 28 may comprise an electrode interface controller 36 that is interconnected to the electrode interface 38, and is operable to control the routing operation of the electrode interface 38. In this regard, a control signal may comprise a digital signal and the electrode interface controller 38 may include digital logic. Further, the power source 32 may be interconnected to the electrode interface 38 to provide power to various digital and analog componentry therein.

It should be appreciated that numerous variations to the embodiments described above may be provided to achieve an output stage for a neurostimulation system that is powered by a single power supply. For example, the description above is directed to a system that utilizes a positive stimulation current and a negative discharge current, but the currents may also be in the opposite direction. Further, although MOS current sources were described, the present invention will also work well using other technologies (e.g., bipolar junction transistors) or other combinations of components. Additionally, although a single output stage 20 was illustrated driving one or more electrodes in an electrode array, it should be appreciated that multiple, independent output stages may be used to drive one or more electrodes in one or more electrode arrays to suit a particular application.

What is claimed is:

1. An output stage for an auditory neurostimulation electrode that is operable to effect a plurality of stimulation and discharge intervals, the output stage comprising:
a stimulation channel coupled to said electrode, wherein a stimulation current may flow therethrough during said stimulation intervals;
a discharge channel coupled to said electrode, wherein a discharge current may flow therethrough during said discharge intervals; and
a controller that is operable to selectively control the flow of current through said stimulation channel and said discharge channel during said stimulation and discharge intervals;
wherein one of said stimulation channel and said discharge channel couples said electrode to a voltage supply, and the other of said stimulation channel and said discharge channel couples said electrode to a reference potential node.

2. An output stage as recited in claim 1, wherein said controller is operable to control the timing of said stimulation and discharge intervals such that said stimulation and discharge intervals are successive.

3. An output stage as recited in claim 1, wherein the amount of charge transferred in said discharge intervals is greater than the amount of charge transferred in said stimulation intervals.

4. An output stage as recited in claim 3, wherein said stimulation current and said discharge current are dependent upon a corresponding physical characteristic of said stimulation channel and said discharge channel.

5. An output stage as recited in claim 4, wherein said stimulation channel and said discharge channel each includes a transistor, and wherein said physical characteristic includes a physical dimension of each transistor.

6. An output stage as recited in claim 1, wherein said discharge current is limited by the potential difference between said electrode and at least one of said reference potential node and the voltage of said voltage supply.

7. An output stage as recited in claim 1, wherein said controller is further operable to selectively adjust the magnitude of said stimulation and discharge currents.

8. An output stage as recited in claim 1, further comprising a charge recovery mechanism that is operable to recover accumulated charges on said electrode.

9. An output stage as recited in claim 8, wherein said charge recovery mechanism includes a resistor that is selectively interconnectable between said electrode and said reference potential node.

10. An output stage as recited in claim 1, wherein said controller includes at least one current mirror.

11. An output stage as recited in claim 10, wherein the amount of charge that flows through said stimulation channel and said discharge channel during said stimulation and discharge intervals is dependent upon a physical dimension of at least one component of said current mirror.

12. An output stage as recited in claim 1, wherein said output stage is interconnected with an electrode interface that is operable to selectively interconnect an output of said output stage to one or more of a plurality of auditory neurostimulation electrodes.

13. An output stage as recited in claim 12, wherein said electrode interface is operable to selectively interconnect said output of said output stage to a first and second set of said plurality of auditory neurostimulation electrodes to effect a plurality of successive stimulation and discharge intervals on said first and second sets of electrodes, wherein said first and second sets are not identical.

14. A method for driving an electrode for auditory neurostimulation, the method comprising:
first transferring a stimulation current between an electrode and one of a voltage supply and a reference potential node; and
second transferring a discharge current between said electrode and the other of said voltage supply and said reference potential node.

15. A method as recited in claim 13, wherein the amount of charge transferred in said first transferring step is less than the amount of charge transferred in said second transferring step.

16. A method as recited in claim 13, further comprising:
limiting the amount of charge transferred in said second transferring step dependent upon the voltage potential on said electrode.

17. A method as recited in claim 13, further comprising:
selectively alternating between said first and second transferring steps to provide auditory neurostimulation to a patient.

18. A method as recited in claim 13, further comprising:
selectively varying the duration of each of said first and second transferring steps.

19. A method as recited in claim 13, further comprising:
selectively varying the amount of charge transferred in at least one of said first and second transferring steps.

20. A method as recited in claim 13, further comprising:
removing accumulated charges from said electrode.

21. A method as recited in claim 13, wherein the amount of charge transferred in the first and second transferring steps is regulated by a current mirror.

22. A method as recited in claim 21, wherein the amount of charge transferred in the first and second transferring steps is dependent upon a physical dimension of at least one component of said current mirror.
